(19) United States
(12) Patent Application Publication
(10) Pub. No.: US 2008/0071335 A1
(43) Pub. Date: Mar. 20, 2008

(54) ANTI-SLIP TREATMENT PAD

(76) Inventor: CHI-HSIN CHEN, Kan ChingLi (TW)

Correspondence Address: LEONG C LEI, PMB # 1008, 1867 YGNACIO VALLEY ROAD, WALNUT CREEK, CA, 94598, US

(21) Appl. No.: 11/532,111

(22) Filed: Sep. 15, 2006

(51) Int. Cl. A61F 7/00

(52) U.S. Cl. 607/112; 607/114

(57) ABSTRACT

An anti-slip treatment pad is enclosed. The treatment pad comprises a band with two ends and having a treatment pad characterized in that the two ends are provided with a fastening unit and the band forms into a circular body by fastening the two ends of the band to each other, and the band is applied onto any part of the body and is fastened by means of the fastening unit to form the anti-slip treatment pad.

### BACKGROUND OF THE INVENTION

(a) Technical Field of the Invention

The present invention related to a hot and cold treatment pad, and in particular, an anti-slip treatment pad.

(b) Description of the Prior Art

Treatment pad is used either as hot or cold treatment pad to soothe exercise injuries or to reduce fever using the cold treatment pad. Generally, these pads do not have a specific securing structure, i.e., these pads are directly applied onto the forehead or applied to the part of the body which is injured. In order to allow the pad to generate its efficiencies, the user needs to keep in a specific position, facilitating the pads to contact with the body, for a specific period. This is not convenience in terms of application.

Taiwanese Patent Publication No. 411843 entitled “Cold and Hot Treatment Ear Hood” and Taiwanese Patent Publication No. 456180, entitled “Bret-type Cold and Hot Band Structure” disclose treatment pad with a specific mounting or securing to the user.

In view of the drawbacks found in conventional treatment pad, it is an object of the present invention to provide an anti-slip treatment pad which solve the above problems and drawbacks.

### SUMMARY OF THE INVENTION

The primary purpose of the present invention is to provide an anti-slip treatment pad comprising a band with two ends and having a treatment pad characterized in that the two ends are provided with a fastening unit and the band forms into a circular body by fastening the two ends of the band to each other, and the band is applied onto any part of the body and is fastened by means of the fastening unit to form the anti-slip treatment pad.

Yet still another object of the present invention to provide an anti-slip treatment pad, wherein the internal and external side of the two ends of the band are provided with a first fastening plate and a second fastening plate.

A further object of the present invention is to provide an anti-slip treatment pad, wherein the two ends of the band are provided with a first fastening element and a second fastening element such that the first and second fastening element fastens to each other to form a circular body.

Still yet another object of the present invention is to provide an anti-slip treatment pad, wherein the band is provided with a plurality of elastic bands corresponding to the treatment pad, and the end portion of each elastic band and the corresponding surface of the circular band are respectively provided with a first and a second fastening plate, such that a user can secure the elastic band onto the circular band.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of the anti-slip treatment pad of the present invention.

FIG. 2 is a cross sectional view of a coiled anti-slip treatment pad of the present invention.

FIG. 3 is a schematic view showing the application of the anti-slip treatment pad of the present invention.

FIG. 4 is an external view of another preferred embodiment of an anti-slip treatment pad of the present invention.

FIG. 5 is a schematic view of another preferred embodiment of an anti-slip treatment pad of the present invention.

FIG. 6 is a schematic view of a further preferred embodiment of an anti-slip treatment pad of the present invention.

### DETAILED DESCRIPTION OF THE PREFERRED EMBODIMENTS

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

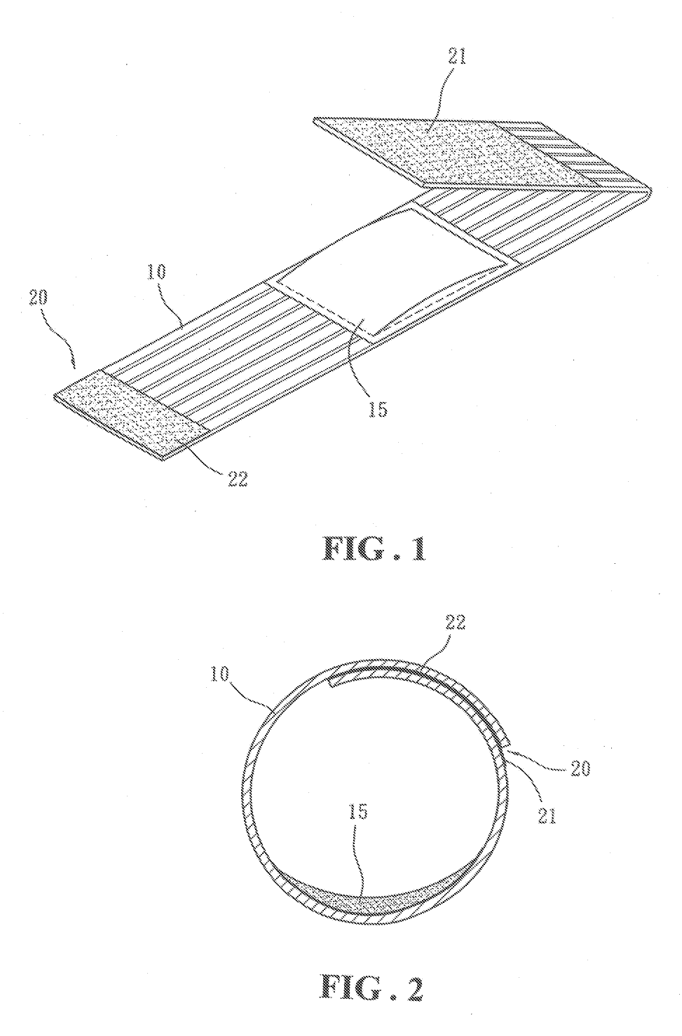

Referring to FIG. 1, there is shown an anti-slip treatment pad for hot and cold treatment on a human body. In accordance with a preferred embodiment, the anti-slip treatment pad comprises a circular band 10, a treatment pad 15 and fastening unit 20. The treatment pad 15 is mounted on the circular band 10 and the fastening unit 20 is mounted at the two ends of the circular band 10 such that the circular band 10 can be formed into a round body by connecting the end to each other.

In accordance with the present invention, and also refer to FIGS. 1 and 2, the circular band 10 is elastic and is being made from a water resistance material, and the treatment pad 15 is mounted at the lateral face and at the center of the circular band 10. The treatment pad 15 can be fixed or detachable from the circular band 10 and can be either a hot treatment pad or a cold treatment pad. The fastening unit 20 is mounted at the interior and exterior face of the two ends of the circular band 10 and is mounted with a first fastening plate 21 and a second fastening plate 22 which could connect or adhere to each other forming into a circular body, as shown in FIG. 2.

The user can apply the circular band 10 directly onto the specific part of the body which requires treatment and the fastening element 20 is used to secure the circular band 10. Thus, in accordance with the present invention, an anti-slip treatment pad is obtained.

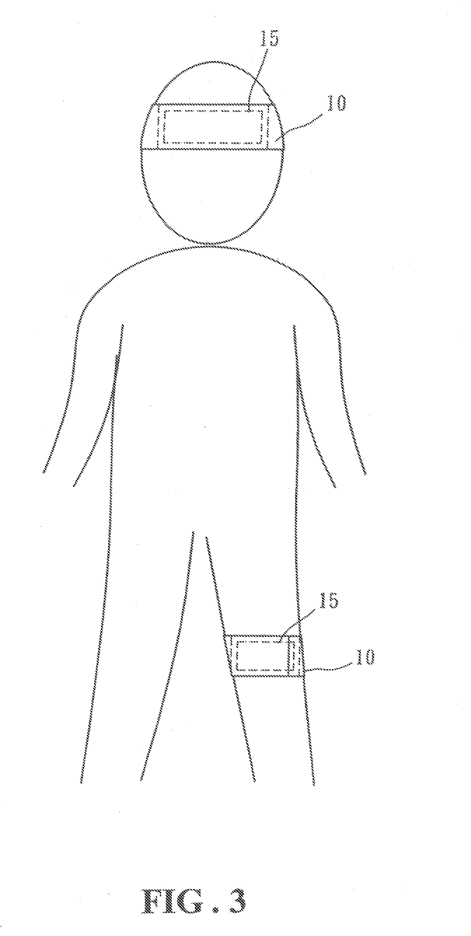

Referring to FIGS. 1, 2 and 3 for actual application, when the part of the body of the user is to be either hot treatment or cold treatment, the circular band 10 surrounds the part of the body such that the treatment pad 15 of the circular band 10 is corresponding to the part of the body. Next, the first and second fastening plate 21 and 22 of the fastening unit 20 are mounted to each other such that the circular band 10 forms into a circular body. Due to the elasticity of the circular band 10, the securing of the anti-slip treatment pad is greatly enhanced and is applicable to any parts of the body.

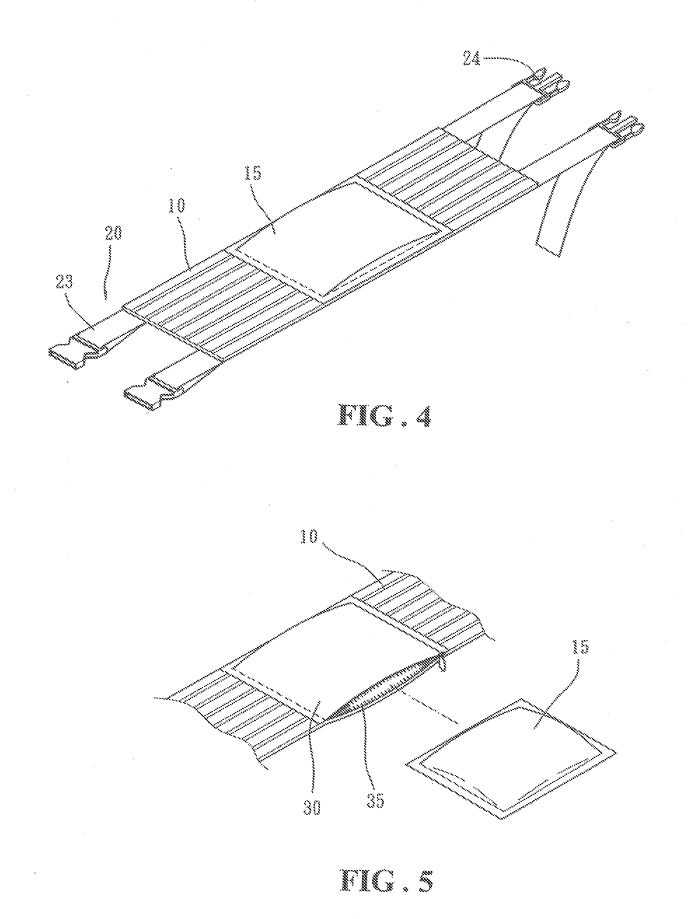

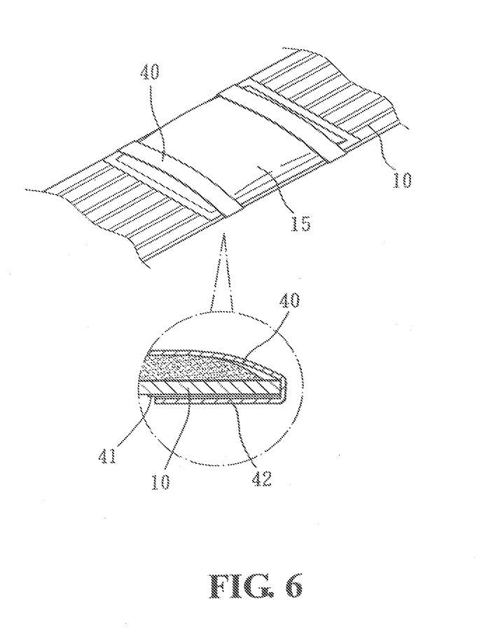

As shown in FIG. 4, there is shown another preferred embodiment of the present invention, the two ends of the circular band 10 is provided with a first fastening element 23 and a second fastening element 24 which could fasten to each other forming into a circular body. This structure, similarly, has anti-slip function and a securing function. As shown in FIG. 5, the center section of the circular band 10 has a holding bag 30 having an opening. The holding bag 30 can hold different treatment pad 15 such that the user can replace a used treatment pad or a different type of treatment pad 15. The opening of the holding bag 30 is provided with a sealing member 35 which is mounted with a zipper or a fastening VELCRO, facilitating the user to withdraw the treatment pad 15. As shown in FIG. 6, the circular band 10 is provided with a plurality of an elastic band 40 which correspond to the treatment pad 15. The end portion of the elastic band 40 and the corresponding surface of the circular band 10 are provided with a first fastening plate 41, and a second fastening plate 42, such that the user can secure the treatment pad 15 to the circular band 10 by means of the elastic band 40. This provides an easy and convenience replacement of a new treatment pad.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. An anti-slip treatment pad comprising a band with two ends and having a treatment pad characterized in that the two ends are provided with a fastening unit and the band forms into a circular body by fastening the two ends of the band to each other, and the band is applied onto any part of the body and is fastened by means of the fastening unit to form the anti-slip treatment pad.

2. The anti-slip treatment pad of claim 1, wherein the band is elastic and being made from a water resistant material.

3. The anti-slip treatment pad of claim 1, wherein the treatment pad is a hot treatment pad or a cold treatment pad.

4. The anti-slip treatment pad of claim 1, wherein the internal and external said of the two ends of the band are provided with a first fastening plate and a second fastening plate.

5. The anti-slip treatment pad of claim 1, wherein the two ends of the band are provided with a first fastening element and a second fastening element such that the first and second fastening element fastens to each other to form a circular body.

6. The anti-slip treatment pad of claim 1, wherein the circular band is provided with a bag with an opening for holding a treatment pad.

7. The anti-slip treatment pad of claim 6, wherein the opening is mounted with a seal element to prevent pad from falling out from the opening.

8. The anti-slip treatment pad of claim 7, wherein the fastening element is a zip or a self-fastening VELCRO, facilitating the withdrawal or insertion of a treatment pad.

9. The anti-slip treatment pad of claim 1, wherein the band is provided with a plurality of elastic bands corresponding to the treatment pad, and the end portion of each elastic band and the corresponding surface of the circular band are respectively provided with a first and a second fastening plate, such that a user can secure the elastic band onto the circular band.
